(19) United States
(12) Patent Application Publication
(10) Pub. No.: US 2019/0051414 A1
(43) Pub. Date: Feb. 14, 2019

(54) SYSTEM AND METHOD FOR VEHICLE-BASED HEALTH MONITORING

(71) Applicant: Faraday&Future Inc., Gardena, CA (US)

(72) Inventors: Anna Angelica Lyubich, Sunnyvale, CA (US); Andrew Wayne Konecki, Pleasanton, CA (US); Carlos John Rosario, San Jose, CA (US)

(21) Appl. No.: 15/713,511

(22) Filed: Sep. 22, 2017

Related U.S. Application Data
(60) Provisional application No. 62/398,838, filed on Sep. 23, 2016.

(51) Int. Cl. G16H 50/30; G06K 9/00; G07C 5/00; G16H 10/60; H04W 4/021; H04W 4/40

(52) U.S. Cl. CPC G16H 50/30; G06K 9/00845; H04W 4/40; G16H 10/60; H04W 4/021; G07C 5/008

(57) ABSTRACT

A vehicle is disclosed. The vehicle may comprise one or more sensors configured to capture occupant information of an occupant of the vehicle and environmental information. The vehicle may also comprise a processor coupled to the one or more sensors. The processor may be configured to associate the captured occupant information and the captured environmental information with an identity of the occupant, and transmit the captured occupant information and the captured environmental information together with the associated identity of the occupant to a third party device.

### CROSS-REFERENCE TO RELATED APPLICATIONS

This application claims the benefit of U.S. Provisional Application No. 62/398,838, filed Sep. 23, 2016, the entirety of which is hereby incorporated by reference.

### TECHNICAL FIELD

The present disclosure relates generally to health monitoring, and more particularly, to methods and systems for vehicle-based health monitoring.

### BACKGROUND

Most people lack of convenient methods for keeping track of their health parameters comprehensively and accurately. Common wearable devices are limited to monitoring a few rudimentary parameters with arguable accuracies. Established medical facilities often provide services to visitors for a practically limited number of times due to the constraint of medical resources and patients' burden. Thus, there is a long-felt need for systems providing convenient, comprehensive, and regular health monitoring, which can lead to better diagnosis or treatment.

Further, some vehicle accidents may be related to driver's health issues. However, due to the destructive nature of accidents, it may be difficult to obtain evidences and conclude causes with existing technologies. In such scenarios, a system which can monitor both the driver's health and the vehicle's status is desired.

### SUMMARY

One aspect of the present disclosure is directed to a vehicle. The vehicle may comprise one or more sensors configured to capture occupant information of an occupant of the vehicle and environmental information. The vehicle may also comprise a processor coupled to the one or more sensors. The processor may be configured to associate the captured occupant information and the captured environmental information with an identity of the occupant, and transmit the captured occupant information and the captured environmental information together with the associated identity of the occupant to a third party device.

Another aspect of the present disclosure is directed to a vehicle-based health monitoring method. The method may be implemented by a vehicle. The method may comprise capturing occupant information of an occupant of the vehicle, capturing environmental information, associating the captured occupant information and the captured environmental information with an identity of the occupant, and transmitting the captured occupant information and the captured environmental information together with the associated identity to a third party device.

Another aspect of the present disclosure is directed to a vehicle. The vehicle may comprise one or more sensors configured to capture one or more health parameters of an occupant of the vehicle and environmental information. The vehicle may also comprise a processor coupled to the one or more sensors. The processor may be configured to determine an identity of the occupant, associate the captured one or more health parameters and the captured environmental information with the determined identity, and transmit the captured one or more health parameters and the captured environmental information together with the associated identity to a third party device.

It is to be understood that the foregoing general description and the following detailed description are exemplary and explanatory only, and are not restrictive of the invention, as claimed.

### BRIEF DESCRIPTION OF THE DRAWINGS

The accompanying drawings, which constitute a part of this disclosure, illustrate several embodiments and, together with the description, serve to explain the disclosed principles.

FIG. 1 is a graphical representation illustrating a vehicle for health monitoring, consistent with exemplary embodiments of the present disclosure.

FIG. 2 is a block diagram illustrating a system for health monitoring, consistent with exemplary embodiments of the present disclosure.

FIG. 3 is a flowchart illustrating a method for health monitoring, consistent with exemplary embodiments of the present disclosure.

### DETAILED DESCRIPTION

Reference will now be made in detail to exemplary embodiments, examples of which are illustrated in the accompanying drawings. The following description refers to the accompanying drawings in which the same numbers in different drawings represent the same or similar elements unless otherwise represented. The implementations set forth in the following description of exemplary embodiments consistent with the present invention do not represent all implementations consistent with the invention. Instead, they are merely examples of systems and methods consistent with aspects related to the invention.

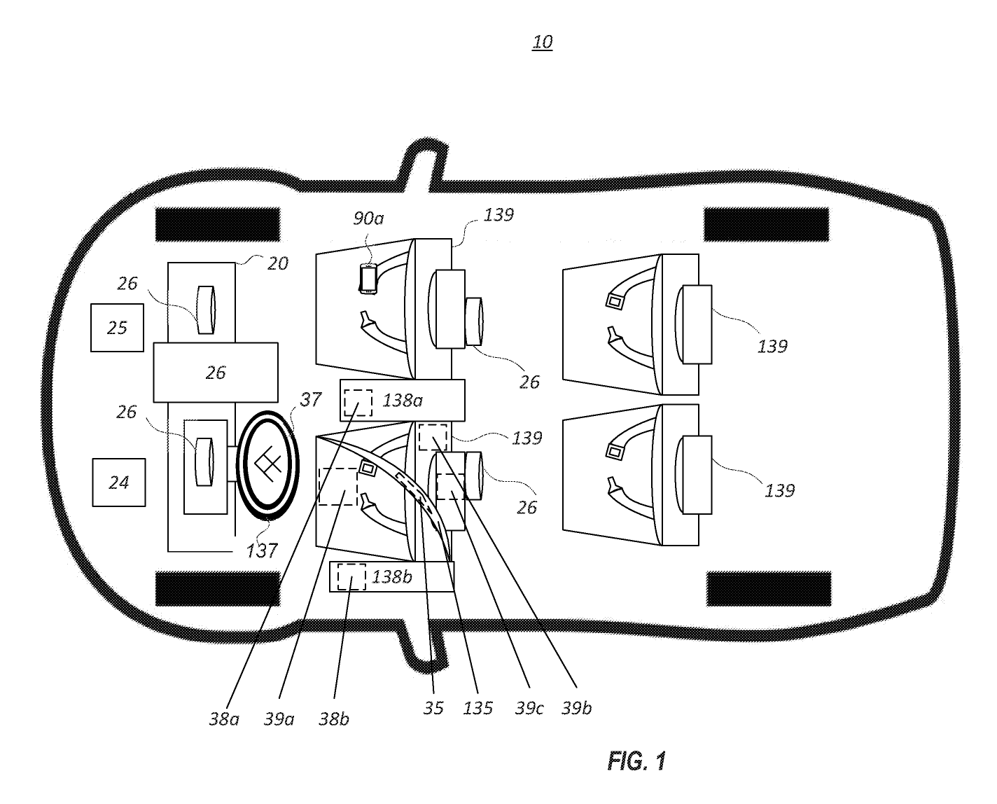

FIG. 1 is a graphical representation illustrating a vehicle 10 for attention monitoring, consistent with exemplary embodiments of the present disclosure. Vehicle 10 may have any body style of an automobile, such as a sports car, a coupe, a sedan, a pick-up truck, a station wagon, a sports utility vehicle (SUV), a minivan, or a conversion van. Vehicle 10 may also embody other types of transportation, such as motorcycles, boats, buses, trains, and planes. Vehicle 10 may be an electric vehicle, a fuel cell vehicle, a hybrid vehicle, or a conventional internal combustion engine vehicle. Vehicle 10 may be configured to be operated by a driver occupying vehicle 10, remotely controlled, and/or autonomous. That is, the methods described herein can be performed by vehicle 10 with or without a driver.

As illustrated in FIG. 1, vehicle 10 may include a number of components, some of which may be optional. Vehicle 10 may have a dashboard 20 through which a steering wheel 137 and one or more user interfaces 26 may project. Steering wheel 137 may include one or more wheel sensors 37 configured to monitor one or more health parameters of an occupant. For example, wheel sensor 37 may include a touch sensor, a pressure sensor, and/or a temperature sensor disposed on the steering wheel. User interface 26 may include one or more interface sensors 36 configured to receive and transmit data, detect and recognize occupants, monitor one or more health parameters of occupants, and/or perform other functions as described below. Vehicle 10 may also include a GNSS (Global Navigation Satellite System (e.g., GPS (Global Positioning System), BeiDou, Galileo) unit 24 disposed in front of steering wheel 137, on the top of the vehicle, or at another location. GNSS unit 24 may be configured to receive signals (e.g., GPS signal), transmit data, and/or determine in real time the location of vehicle 10. Vehicle 10 may also include a detector unit 25 disposed in front of a passenger seat, at another location within the vehicle, or on an exterior of the vehicle. Detector unit 25 may be configured to monitor information of the surrounding environment. For example, detector 25 may include a camera mounted on top of the vehicle and configured to capture images of the outside environment. Vehicle 10 may also have one or more seats 139, such as front seats and back seats configured to accommodate occupants. Each seat may comprise one or more seat sensors 39 (e.g., 39a, 39b, and 39c) configured to measure one or more health parameters of an occupant. For example, the driver's seat may comprise sensor 39a in the bottom cushion of the seat, sensor 39b in the back cushion of the seat, and sensor 39c in the headrest of the seat. Similarly, though not shown in this figure, any other seat may comprise such sensors. Vehicle 10 may also include one or more consoles 138, e.g., a center console 138a disposed between front seats and a side console 138b disposed on the other side of the driver's seat or on the driver's door. Each console may have various configurations and shapes. Each console may comprise one or more console sensors, such as console sensor 38a inside console 138a and console sensor 38b inside console 138b, configured to monitor one or more health parameters of occupants. The console sensors may have various configurations and shapes, e.g., a handle shape. Similarly, though not shown in this figure, such consoles may be disposed at other locations, for example next to other seats in the vehicle. In addition, more user interfaces 26 may be disposed in the vehicle, for example, at the back of the front seats and facing the back seats. Each seat may also be equipped with a seat belt 135. Seat belt 135 may have various configurations, such as comprising a lap belt and a shoulder belt. For simplicity, only a shoulder belt is shown in this figure. Seat belt 135 may comprise a belt sensor 35 configured to monitor one or more health parameters of an occupant. The positions of the various components of vehicle 10 in FIG. 1 are merely illustrative and are not limited as shown in the figure. Components shown in dash lines may be embedded in other components.

In some embodiments, user interface 26 may be configured to receive inputs from users or devices and transmit data. For example, user interface 26 may have a display including an LCD, an LED, an OLED, a plasma display, or any other type of display, and provide a graphical user interface (GUI) presented on the display for user input and data display. User interface 26 may further include speakers or other voice playing devices. User interface 26 may further include input devices, such as a touchscreen, a keyboard, a mouse, a microphone, and/or a tracker ball, to receive a user input. User interface 26 may also connect to a network to remotely receive instructions or user inputs. Thus, the input may be directly entered by a current occupant, captured by interface 26, or received by interface 26 over the network. The input may be associated with monitoring one or more health parameters of a certain body part. The input may be received over an augmented reality or virtual reality presented on user interface 26, for example, via touching a body part presented as a virtual reality. User interface 26 may further include a housing having grooves containing the input devices. User interface 26 may be configured to provide internet access, cell phone access, and/or in-vehicle network access, such as Bluetooth™, CAN bus, or any other vehicle bus architecture protocol that may be used to access features or settings within vehicle 10. User interface 26 may be further configured to display or broadcast other media, such as images, videos, and maps.

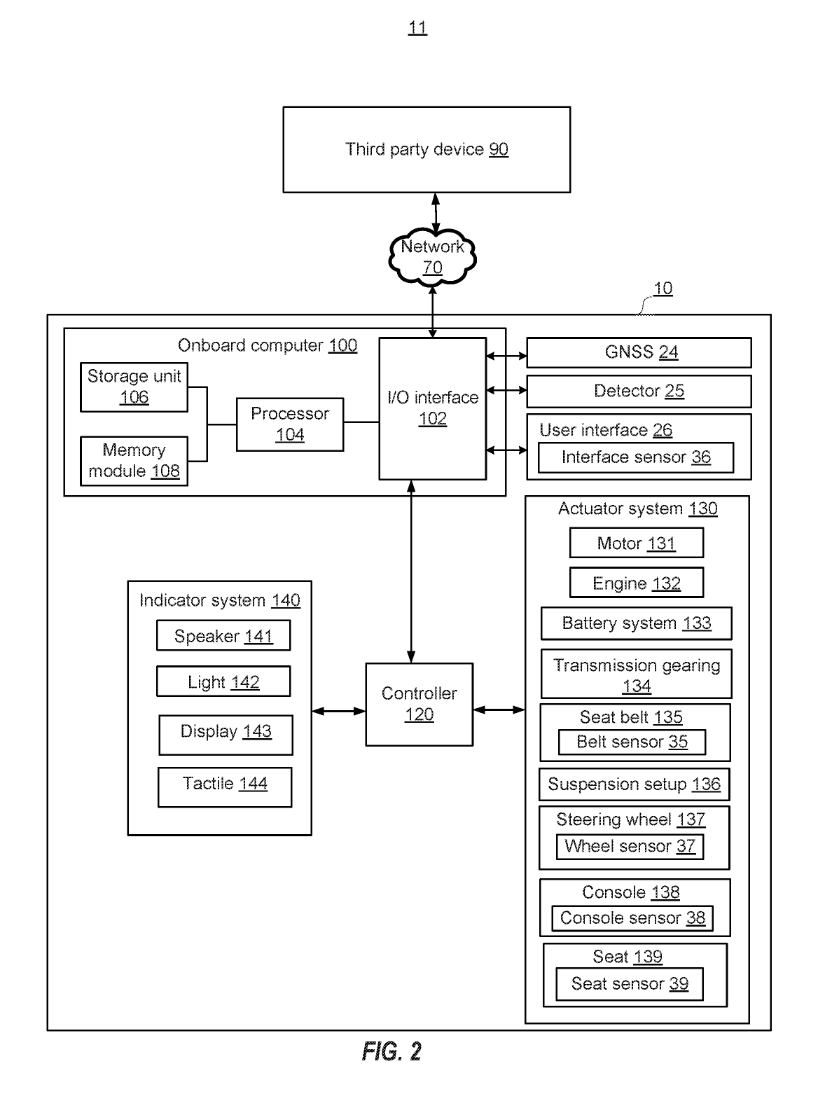

In some embodiments, user interface 26 may also be configured to receive user-defined settings. For example, user interface 26 may be configured to receive occupant profiles including, for example, an age, a gender, a driving license status, an ADAS license status, a frequent destination, a store reward program membership, a frequently purchased item, favorite food, medical history, medical profile, doctor information, and etc. In some embodiments, user interface 26 may include a touch-sensitive surface configured to receive biometric data (e.g., detect a fingerprint of an occupant). The touch-sensitive surface may be configured to detect the ridges and furrows of a fingerprint based on a change in capacitance and generate a signal based on the detected fingerprint, which may be processed by an onboard computer described below with reference to FIG. 2. The onboard computer may be configured to compare the signal with stored data to determine whether the fingerprint matches any recognized occupant. The onboard computer may also be able to connect to the Internet, obtain data from the Internet, and compare the signal with obtained data to identify the occupants. User interface 26 may be configured to include biometric data into a signal, such that the onboard computer can identify the person generating an input. User interface 26 may also compare a received voice input with stored voices to identify the person generating the input. Furthermore, user interface 26 may be configured to store data history accessed by the identified person. In some embodiments, information about an occupant (e.g., information indicating that a particular occupant is located in vehicle 10, information indicating the health of an occupant) can be transmitted to another vehicle (e.g., a vehicle associated with a friend or relative of the occupant).

In some embodiments, user interface 26 may be configured to present augmented reality or virtual reality contents. In one example, virtual meeting applications such as WebEx™, Lync™, and FaceTime™ for conferencing on the road may be use via user interface 26. In another example, user interface 26 may present trip planning or route organization by overlaying configurable destinations with a map. In such presentations, calendar, meetings, and events schedule as icons may be integrated to allow free selection and drag-and-drop. User interface 26 may also present GIS (Geographic Information System) information to visualize, query, analyze, and interpret data, and to present relationships, patterns, and trends. Further, 3D virtual mapping (that is, 3D imaging for city buildings and road map) can be displayed via user interface 26, so that users can, for example, select a building, rotate it, check what is behind the building, or select a building to obtain more information about the building. In yet another example, user interface 26 may connect to in-home security camera(s) while on the road, to remotely monitor home security.

In some embodiments, interface sensor 36 may be configured to detect and/or recognize occupants of vehicle 10. In one example, interface sensor 36 may obtain identifications from occupants' cell phones. In another example, interface sensor 36 may embody as a camera configured to capture images of an occupant. In some embodiments, visually captured videos or images by interface sensor 36 may be used in conjunction with an image recognition software, such that the software may distinguish a person from inanimate objects, may recognize the person based on physical appearances or traits, and may recognize body parts such as hands and eyes. The image recognition software may include a facial recognition software configured to match a captured occupant with stored profiles to identify the occupant. The image recognition software may also identify relative positions of the body parts in any space system, for example positions of the hands relative to a steering wheel, lines of sight of the eyes, and etc. In some embodiments, more than one sensor may be used in conjunction to detect and/or recognize the occupant(s). For example, interface sensor 36 may include a camera and a microphone to capture images and voices, and a processor may use the captured images and voices as filters to identify the occupant(s) based on the stored profiles. Similarly, LIDAR may perform a similar function as the camera described above.

In some embodiments, interface sensor 36 may include one or more electrophysiological sensors for encephalography-based health monitoring. For example, a fixed interface sensor 36 may detect electrical activities of brains of the occupant(s), monitor the brain activities, and/or convert the electrical activities to signals. The onboard computer may control one or more components of vehicle 10 based on the signals. Interface sensor 36 may also be detachable and head-mountable, and may detect the electrical activities when worn by the occupant(s).

The sensors of vehicle 10, including belt sensor 35, interface sensor 36, wheel sensor 37, console sensor 38, seat sensor 39, and detector 25, may be collectively referred to as vehicle sensors. The vehicle sensors may have various configurations to monitor one or more health parameters of one or more vehicle occupants passively or actively. The vehicle sensors may include a camera, a microphone sound detection sensor, an infrared sensor, a weight sensor, a radar, an ultrasonic sensor, a LIDAR sensor, a touch sensor, a wireless sensor, etc. The passive monitoring may refer to enabling monitoring upon a user signal or command. The active monitoring may refer to enabling monitoring without an explicit user command. The one or more health parameters may include, for example, blood glucose level, sweat compositions, heart rate, body temperature, blood pressure, and similar indications.

Vehicle 10 may be in communication with one or more third party devices 90 (e.g., mobile communication device 90a). Third party devices 90 may include a smart phone, a tablet, a computer, a server, a health practitioner terminal device, a different vehicle, a wearable device, such as a smart watch or Google Glass™, and/or similar devices. Third party devices 90 may be configured to connect to a network, such as a nationwide cellular network, a local wireless network (e.g., Bluetooth™ or WiFi), and/or a wired network. Third party devices 90 may also be configured to access apps and websites of third parties, such as iTunes™, Pandora™, Google™, Facebook™, and Yelp™.

In some embodiments, third party devices 90, e.g., mobile communication device 90a may be carried by or associated with one or more occupants in vehicle 10. Vehicle 10 may be configured to determine the presence of specific people based on a digital signature or other identification information from mobile communication device 90a. For instance, an onboard computer of vehicle 10 may be configured to relate the digital signature to stored profile data including the person's name and the person's relationship with vehicle 10. The digital signature of mobile communication device 90a may include a determinative emitted radio frequency (RF) or a GNSS (e.g., GPS, BeiDou, Galileo) tag. Mobile communication device 90a may be configured to automatically connect to or be detected by vehicle 10 through a network 70, e.g., Bluetooth™ or WiFi, when positioned within a proximity (e.g., within vehicle 10).

In some embodiments, third party devices 90 may be associated with a health practitioner. Third party devices 90 may be a computer, a tablet, a wearable device, or etc. For example, third party devices 90 may include a computer at a doctor's office, a tablet carried by a nurse, a mobile phone of a rescuer, and the like.

FIG. 2 is a block diagram illustrating a system 11 for health monitoring, consistent with exemplary embodiments of the present disclosure. System 11 may include a number of components, some of which may be optional. As illustrated in FIG. 2, system 11 may include a vehicle 10 connected to a third party device 90 through network 70. Vehicle 10 may include a specialized onboard computer 100, a controller 120, an actuator system 130, an indicator system 140, a GNSS unit 24, a detector 25, and one or more user interfaces 26. Each user interface 26 may include one or more interface sensors 36. Onboard computer 100, actuator system 130, and indicator system 140 may all couple to controller 120. User interface 26, detector 25, and GNSS unit 24 may all couple to onboard computer 100. Onboard computer 100 may comprise, among other things, an I/O interface 102, a processor 104, a storage unit 106, and a memory module 108. The above units of system 11 may be configured to transfer data and send or receive instructions between or among each other. Storage unit 106 and memory module 108 may be non-transitory and computer-readable memories storing instructions that, when executed by processor 104, cause system 11 or vehicle 10 to perform one or more methods described in this disclosure. Onboard computer 100 may be specialized to perform the methods and steps described below.

I/O interface 102 may include connectors for wired communications, wireless transmitters and receivers, and/or wireless transceivers for wireless communications. The connectors, transmitters/receivers, or transceivers may be configured for two-way communication between onboard computer 100 and various components of system 11. I/O interface 102 may send and receive operating signals to and from third party device 90. I/O interface 102 may send and receive the data between each of the devices via communication cables, wireless networks, or other communication mediums. For example, mobile communication device 90a may be configured to send and receive signals to I/O interface 102 via a network 70. Network 70 may be any type of wired or wireless network that may facilitate transmitting and receiving data. For example, network 70 may be a nationwide cellular network, a local wireless network (e.g., Bluetooth™ or WiFi), and/or a wired network.

Third party devices 90 may include mobile phones, smart phones, computers, laptops, pads, servers, one or more vehicles, processors of third parties, and/or other terminals. Third party device 90 may provide access to or receive contents and/or data (e.g., maps, health information, traffic, store locations, weather, instruction, command, user input). Third party devices 90 may be directly accessible by onboard computer 100, via I/O interface 102, according to respective authorizations of the user. For example, users may allow onboard computer 100 to receive third party contents by configuring settings of accounts with third party devices 90 or settings of mobile communication devices 90a. In one example, third party device 90 may be a terminal device, e.g., a computer, associated with a health practitioner and may receive health parameters monitored by the vehicle sensors. Third party devices 90 may also transmit instructions or other data to the vehicle sensors and user interfaces of vehicle 10.

Processor 104 be configured to receive signals and process the signals to determine a plurality of conditions of the operation of vehicle 10 (e.g., operations of indicator system 140 through controller 120, and if vehicle 10 is crashed or involved in an accident), a plurality of conditions of a vehicle occupant (e.g., health condition based on monitored health parameters), and/or a plurality of outside environment (e.g., a location of the vehicle, a restaurant or location associated with the vehicle, or a view of the surrounding). Processor 104 may also be configured to generate and transmit command signals, via I/O interface 102, in order to actuate the devices in communication.

In some embodiments, processor 104 may be configured to determine the presence of people within an area, such as occupants of vehicle 10. Processor 104 may be configured to determine the identity of the occupants through a variety of mechanisms. For example, processor 104 may be configured to determine the presence of specific people based on a digital signature from mobile communication device 90a. For instance, processor 104 may be configured to relate the digital signature to stored data including the person's name and the person's relationship with vehicle 10. The digital signature of communication device 90a may include a determinative emitted radio frequency (RF), GPS, Bluetooth™, or WiFi unique identifier. Processor 104 may also be configured to determine the presence of people within vehicle 10 by GNSS tracking software of mobile communication device 90a. In some embodiments, vehicle 10 may be configured to detect mobile communication device 90a when mobile communication device 90a connects to local network 70 (e.g., Bluetooth™ or WiFi).

In some embodiments, processor 104 may also be configured to recognize occupants of vehicle 10 by receiving inputs with user interface 26. For example, user interface 26 may be configured to receive direct inputs of the identities of the occupants. User interface 26 may also be configured to receive biometric data (e.g., fingerprints) from occupants. Processor 104 may be further configured to recognize occupants by facial recognition software used in conjunction with the vehicle sensors.

In some embodiments, processor 104 may be configured to access and collect sets of data related to the people within the area in a number of different manners. Processor 104 may be configured to store the sets of data in a database. In some embodiments, processor 104 may be configured to access sets of data stored on mobile communication device 90a, such as apps, audio files, text messages, notes, messages, photos, and videos. Processor 104 may also be configured to access accounts associated with third party devices 90. Processor 104 may be configured to receive data directly from occupants, for example, through access of user interface 26. Processor 104 may also be configured to receive data from the history of previous inputs of the occupant into user interface 26.

Storage unit 106 and/or memory module 108 may be configured to store one or more computer programs that may be executed by onboard computer 100 to perform functions of system 11. For example, storage unit 106 and/or memory module 108 may be configured to store biometric data detection and processing software configured to determine the identity of people based on fingerprint(s), and store image recognition software configured to relate images to identities of people. Storage unit 106 and/or memory module 108 may be further configured to store data and/or look-up tables used by processor 104. For example, storage unit 106 and/or memory module 108 may be configured to include data related to individualized profiles of people related to vehicle 10. In some embodiments, storage unit 106 and/or memory module 108 may store the data and/or the database described in this disclosure.

Controller 120 is connected to one or more actuator systems 130 in the vehicle and one or more indicator systems 140 in the vehicle. One or more actuator systems 130 may include, but are not limited to, a motor 131 or engine 132, a battery system 133, a transmission gearing 134, one or more seat belts 135, a suspension setup 136, a steering wheel 137, one or more consoles 138, and one or more seats 139. Each seat belt 135 may include a belt sensor 35. Steering wheel 137 may include one or more sensors 37. Each console 138 may comprise one or more console sensors 38. Each seat 139 may comprise one or more seat sensors 39. Some of these components may be optional. For example, an electric vehicle may not include engine 132. The onboard computer 100 can control, via controller 120, one or more of these actuator systems 130 during vehicle operation, for example, to open or close one or more of the doors of the vehicle, to control the vehicle during autonomous driving or parking operations, and etc.

One or more indicator systems 140 may include, but are not limited to, one or more speakers 141 in the vehicle (e.g., as part of an entertainment system in the vehicle or part of user interface 26), one or more lights 142 in the vehicle, one or more displays 143 in the vehicle (e.g., as part of a control or entertainment system in the vehicle), and one or more tactile actuators 144 in the vehicle (e.g., as part of a steering wheel or seat in the vehicle). Display 143 may include a touch screen/display that provides an interactive interface.

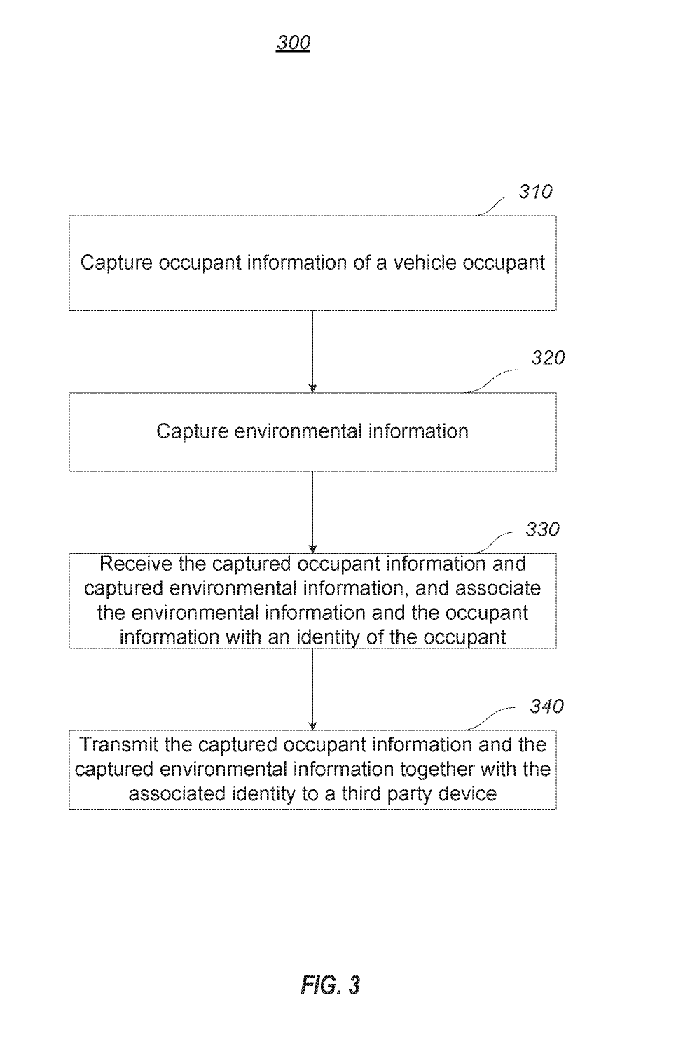

FIG. 3 is a flowchart illustrating a method 300 for health monitoring, consistent with exemplary embodiments of the present disclosure. Method 300 may include a number of steps and sub-steps, some of which may be optional. The steps or sub-steps may also be rearranged in another order. For example, step 310 and step 320 may be implemented in any order or simultaneously.

In Step 310, one or more components of system 10 or 11 may capture occupant information. The occupant information may include an occupant identity and one or more health parameters. The occupant information may be captured by a non-contact sensor, e.g., interface sensor 36 of user interface 26 described above, and/or a contact sensor, e.g., wheel sensor 37, console sensor 38, seat sensor 39, or belt sensor 35 described above. The captured occupant information may be transmitted to processor 104, which may determine the occupant identity. For example, one or more of the vehicle sensors may detect a number of occupants in vehicle 10 and capture their associated voices, images, gestures, and the like. Such information may be transmitted to processor 104 to determine their identities. For another example, interface sensor 36 may include a cellphone detection sensor that detects the occupants according to mobile communication device 90a connected to a local wireless network (e.g., Bluetooth™) of vehicle 10, and transmit the detected information to processor 104. For another example, user interface 26 may detect the occupants according to manual entry of data into vehicle 10, e.g., occupants selecting individual names through user interface 26, and transmit the detected information to processor 104. Processor 104 may also receive biometric data (e.g., fingerprint, palm print data, or iris data) of a vehicle occupant captured by user interface 26, wheel sensor 37, console sensor 38, seat sensor 39, or belt sensor 35. For another example, interface sensor 36, console sensor 38, or seat sensor 39 may include cameras that capture images of occupants, microphones that capture voices of occupants, and/or weight sensors that capture weights of objects on the vehicle seats. Based on the captured data, processor 104 may determine each occupant in vehicle 10.

In some embodiments, one or more components of system 10 or 11 may determine each occupant's identity, by executing a software such as an image recognition software, a voice recognition software, or a weight recognition software, based on the received data from interface sensor 36, wheel sensor 37, console sensor 38, seat sensor 39, and/or belt sensor 35. For example, interface sensor 36 may detect a digital signature or other identification information from mobile communication devices that occupants carry, and processor 104 may determine the occupants' identifies based on the digital signatures. Processor 104 may access, collect, and update sets of data related to each occupant in vehicle 10. Processor 104 may determine whether the determined occupants have stored profiles. Processor 104 may also access sets of data stored on mobile communication device 90a or other third party devices 90 to update the stored profile(s). If an occupant does not have a stored profile, processor 104 may generate a profile based on the accessed data. Each profile may include information such as age, gender, driving license status, driving habit, frequent destination, favorite food, shopping habit, enrolled store reward program, and the associated item(s). Based on the combination of the sensor input and the stored profiles, processor 104 may determine each occupant's identity.

The one or more health parameters may be captured by one or more of the vehicle sensors. In some embodiments, the blood pressure of a vehicle occupant may be measured by wheel sensor 37, console sensor 38, seat sensor 39, and/or belt sensor 35. The blood pressure may be measured continuously or at configurable time periods as long as the occupant remains in the vehicle. The blood pressure may be measured via contact between skin and a piezoelectric of the sensor. The piezoelectric may be disposed on a sensor exterior, e.g., on a surface of a wheel, a handle, a seat, a belt, or the like. The blood pressure measurement method is not limited to the example above, and may include any other method.

In some embodiments, the heart rate of a vehicle occupant may be measured by wheel sensor 37, console sensor 38, seat sensor 39, and/or belt sensor 35. For example, any of the vehicle sensors may include a pulse oximeter probe configured to contact an occupant's skin, as the heart rate may be measured with plethysmograph. The heart rate may be measured continuously or at configurable time periods as long as the occupant remains in the vehicle. The heart rate measurement method is not limited to the example above, and may include any other method.

In some embodiments, the body temperature of a vehicle occupant may be measured by interface sensor 36, wheel sensor 37, console sensor 38, seat sensor 39, and/or belt sensor 35. For example, interface sensor 36 may include an infrared sensor configured to capture an infrared image of the occupant and determine the body temperature based on the captured image. For another example, any of the vehicle sensors may include a temperature sensor configured to measure an occupant's temperature through a direct contact with the occupant. The body temperature may be measured continuously or at configurable time periods as long as the occupant remains in the vehicle. The body temperature measurement method is not limited to the example above, and may include any other method.

In some embodiments, the sweat composition, e.g., sodium levels, of a vehicle occupant may be measured by interface sensor 36, wheel sensor 37, console sensor 38, seat sensor 39, and/or belt sensor 35. For example, interface sensor 36 may include an infrared sensor configured to expose a portion of the occupant's skin to an infrared illumination and remotely sense chemical compositions of the sweat. For another example, any of the vehicle sensors may include a chemical sensor probe configured to measure an occupant's sweat composition through a direct contact with the occupant's skin, e.g., the chemical sensor may be disposed on a handle for sweaty palms. The sweat composition may be measured continuously or at configurable time periods as long as the occupant remains in the vehicle. The sweat composition measurement method is not limited to the example above, and may include any other method.

In some embodiments, the blood composition of a vehicle occupant may be measured by wheel sensor 37, console sensor 38, seat sensor 39, and/or belt sensor 35. For example, any of the vehicle sensors may include an infrared sensor configured to expose a portion of the occupant's skin, e.g., an ear lobe or a webbing connecting a thumb and an index finger, to an infrared illumination and determine the glucose level in the blood through infrared spectroscopy. The sweat composition may be measured continuously or at configurable time periods as long as the occupant remains in the vehicle. The sweat composition measurement method is not limited to the example above, and may include any other method.

In some embodiments, the muscle tension of a vehicle occupant may be measured by console sensor 38, seat sensor 39, and/or belt sensor 35. For example, belt sensor 35 may measure muscle tension across the occupant's chest. The muscle tension may be measured continuously or at configurable time periods as long as the occupant remains in the vehicle. The muscle tension measurement method is not limited to the example above, and may include any other method.

In some embodiments, the eyes of a vehicle occupant may be monitored by interface sensor 36. For example, interface sensor 36 may include a camera configured to capture images of the eyes to help diagnose cataracts or blindness. The eyes may be measured continuously or at configurable time periods as long as the occupant remains in the vehicle. The eye measurement method is not limited to the example above, and may include any other method.

In some embodiments, the internal health of a vehicle occupant may be monitored by interface sensor 36, console sensor 38, seat sensor 39, and/or belt sensor 35. Any of the vehicle sensors may include a radar, ultrasound, or similar sensor configured to scan the occupant's body and organs and obtain corresponding results, such as medical images. For example, belt sensor 35 may be disposed across a waits and configured to scan gastrointestinal organs. For another example, seat sensor 39 may measure a weight of an occupant. For yet another example, console sensor 38 may measure the body fat of the occupant through bioelectrical impedance analysis, or interface sensor 36 may measure the body fat through near-infrared interactance. The internal health may be measured continuously or at configurable time periods as long as the occupant remains in the vehicle. The internal health measurement method is not limited to the example above, and may include any other method.

At step 320, one or more components of system 10 or 11 may capture environmental information. The environmental information may include, for example, a location of vehicle 10, a condition of vehicle 10, and/or a condition of the environment outside vehicle 10. The location of vehicle 10 may include a past, a present, or a future location of vehicle 10 and any associated locations. The location may be captured by GNSS unit 24 described above. For example, if vehicle 10 stopped at a parking lot of 100 Main St., corresponding to an address of a fast food restaurant. The fast food restaurant may be included in in the environmental information as an associated location. The condition of vehicle 10 may include, for example, if the vehicle is operating normally, standby, parked, involved in an accident, crashed, and etc. The condition of the environment outside vehicle 10 may include, for example, an image of the surrounding, an event taking place near vehicle 10 (e.g., an accident close to close to vehicle 10, but not involving vehicle 10), and etc. The condition of the environment may be captured by detector 25 described above.

At step 330, one or more components of system 10 or 11 may receive the captured occupant information and captured environmental information, and associate the environmental information and the occupant information with the identity of the occupant. In some embodiments, one or more components of system 10 or 11 may associate the environmental information with the occupant information to obtain combined information.

For example, processor 104 may associate the monitored health parameters, such as the weight data, blood pressure, or body fat data, and one or more locations that vehicle 10 stopped by with the identity of the occupant. The association may be performed separately for the health parameters and the locations.

For another example, processor 104 may associate the monitored health parameters with one or more locations that vehicle 10 stopped by. The combined information may include a time line indicating visits to certain restaurants (e.g., fast food joints) at various days together with measurements of the weight, blood pressure, or body fat also associated with the days. Combing or associating the monitored health parameters and the one or more locations may be performed by the vehicle at Step 330 or by a third party device at Step 340.

In some embodiments, vehicle 10 may obtain dietary information of the vehicle occupant based on the one or more locations. For example, if the vehicle occupant drives to a fast food restaurant and pays with a mobile phone payment system (that is, an exemplary third party device 90), the vehicle may capture the restaurant name and the bill payment including ordered food as the environmental information. The vehicle may also obtain nutrition facts of the ordered food as part of the environmental information from network database or other sources.

In some embodiments, processor 104 may provide statistical analysis of the data of the combined information, for example, a correlation between the body fat and the restaurant visits or nutrition intakes associated with the restaurant visits. The combined information can help health practitioners to identify health threats. For example, a doctor may utilize the combined information and apply professional knowledge to determine health promotion suggestions, e.g., reducing visits to the fast food restaurants.

For another example, processor 104 may associate the monitored health parameters and a condition of vehicle 10 with the identity of the occupant. For yet another example, processor 104 may associate the monitored health parameters, such as the heart rate, with a condition of vehicle 10 by one or more factors (e.g., time). Combing or associating the monitored health parameters and the vehicle condition may be performed by the vehicle at Step 330 or by a third party device at Step 340. The combined information may include a time line indicating heart rate measurements taken every second of a driver who has a heart-related medical history and the vehicle's condition (e.g., normal operation and crash) associated with the same time line. Thus, even if the vehicle is involved in an accident rendering the driver unconscious, a memory of the vehicle or a third party device may have recorded the driver's heart rate measurements every second before and after the accident, if the associated measurement hardware are still functioning properly. The combined data can help determine if the heart condition was a cause of the accident. More importantly, emergency responders can access such data and other health information even before arriving at the scene, which saves vital time for the rescue.

For another example, processor 104 may associate the monitored health parameters and the condition of the environment outside the vehicle with the identity of the occupant. The condition of the environment may include visual images as seen by the occupant. For yet another example, processor 104 may associate the monitored health parameters, such as the heart rate data, with the condition of the environment outside the vehicle by one or more factors (e.g., time or location). Combing or associating the monitored health parameters and the condition of the environment may be performed by the vehicle in Step 330 or by a third party device at Step 340. The combined information may include a time line indicating heart rate measurements taken at various time stamps of a driver and the environment outside the vehicle, such as images takes at the same time stamps. Through monitoring of the driver's eyes, processor 104 may determine a line of sight and viewing cone of the driver. Through detector 25, processor 104 may capture an image as viewed by the driver in real time. Processor 104 may analyze the image and determine an expected heart rate response of an ordinary person, and adjust the measured heart data by the analysis result. That is, capturing the occupant information may comprise capturing a health parameter of the occupant, and the processor may adjust the captured health parameter based on a baseline reading of the health parameter of an average person in response to the captured visual data. For example, if the road is on a cliff edge or it is raining heavily, an ordinary person would have an increased heart rate, based on which processor 104 may adjust the measured heart rate measurement or increase a threshold for determining an elevated heart rate. Compare to results of traditional wearable devices, the combined information would more accurately reflect health conditions of a user, and thus would be more useful to a health practitioner in determining the user's health.

At step 340, one or more components of system 10 or 11 may transmit the captured occupant information and the captured environmental information together with the associated identity to a third party device. The third party device may combine or associate the captured occupant information and the captured environmental information as described above. The third party device be associated with a health practitioner.

Alternatively, if the captured occupant information and the captured environmental information are combined or associated at Step 330, the one or more components of system 10 or 11 may transmit the combined information to a third party device. For example, processor 104 may transmit the combined information to a computer or another terminal at a health practitioner's office. The combined information can help diagnose diseases and/or render treatments to individual. The disclosed system can lower costs for the public health system since regular health monitoring can be achieved outside the medical facilities. Alternatively, processor 104 may transmit the combined information to the vehicle user's terminal, mobile device 90a, or any other third party device.

In some embodiments, any of the data or information described herein, traffic information such as accidents, and driving information such as the vehicle manual, can be presented at user interface 26 as an augmented reality or virtual reality.

In some embodiments, the occupant information, the environmental information, and/or the combined information can be presented as an augmented reality or virtual reality. For example, the monitored health parameters may be shown on an anatomical body image on user interface 26. A user can select a part of the body to be monitored or configure additional information.

The described methods and systems may form a “Vehicle Health Telemetry System.” A variety of health parameters may be monitored. For example, an occupant's temperature may be monitored for fever or early sickness detection. Shortness of breath (SoB) may be measured via seat belt strain sensors, cameras, or interior LIDAR sensors. Blood pressure or heart rate may be measured by a steering wheel, seat belt, seat, metallic handles, or LIDAR sensors. Gastrointestinal conditions may be measured via seat belt strain, acoustics, or ultrasound sensors. Diagnostics based on sweat analysis, cholesterol levels, or glucose levels, for various diseases such as early on-set diabetes may be performed by the vehicle sensors and processor. Visual and eye tracking may be performed by the vehicle sensors to inspect passenger behavior, or diagnose eye degenerative diseases or cataracts. Body scanning may be performed by ultrasound sensors for internal body diagnostics for stroke, heart attack, stone, or bacterial infections.

In some embodiments, the above-described systems and methods can be applied to vehicles in a platoon. Vehicles traveling in a platoon may travel in a formation with small separations, and accelerate and brake together. The disclosed systems and methods can help monitor health parameters of platoon vehicle drivers.

It should be appreciated that in some embodiments a machine learning algorithm can be implemented such as an as a neural network (deep or shallow, which may employ a residual learning framework) and be applied instead of, or in conjunction with another algorithm described herein to solve a problem, reduce error, and increase computational efficiency in performing the above disclosed methods. For example, deep learning methods may be implemented to increase the accuracy and/or efficiency of determining occupant attributes including, but not limited to health parameters and occupant identities. Such learning algorithms may implement a feedforward neural network (e.g., a convolutional neural network) and/or a recurrent neural network, with supervised learning, unsupervised learning, and/or reinforcement learning. In some embodiments, backpropagation may be implemented (e.g., by implementing a supervised long short-term memory recurrent neural network, or a max-pooling convolutional neural network which may run on a graphics processor). Moreover, in some embodiments, unsupervised learning methods may be used to improve supervised learning methods. Moreover still, in some embodiments, resources such as energy and time may be saved by including spiking neurons in a neural network (e.g., neurons in a neural network that do not fire at each propagation cycle).

The above described methods and systems are more advantageous than existing technologies in many aspects. For example, most of existing health monitoring takes place at a doctor's office, which is limited in time and only provides health data at point of time. If a data pattern has to be accumulated over a period of time, the patient has to visit the doctor's office many times. Further, wearable devices often measure a limited number of simple features such as pulse rates and also lack a complete picture of the health conditions. By contrast, the disclosed methods and systems take advantage of the considerable time spent in vehicles to monitor a variety of health parameters of vehicle occupants, relate them to the environmental information, and provide such results to health practitioners, so that better diagnosis and care can be rendered. The provided results may more accurately reflect the person's health parameter history and correlations with his behavior. In addition, the health parameters can be captured noninvasively and inconspicuously, providing great convenient to the user. For example, the glucose measurement and/or the sweat composition measurement can monitor and forewarn a potential diabetic disease, by spending some time in the vehicle than taking multiple measurements by traditional methods.

A person skilled in the art can further understand that, various exemplary logic blocks, modules, circuits, and algorithm steps described with reference to the disclosure herein may be implemented as specialized electronic hardware, computer software, or a combination of electronic hardware and computer software. For examples, the modules/units may be implemented by one or more processors to cause the one or more processors to become one or more special purpose processors to executing software instructions stored in the computer-readable storage medium to perform the specialized functions of the modules/units.

The flowcharts and block diagrams in the accompanying drawings show system architectures, functions, and operations of possible implementations of the system and method according to multiple embodiments of the present invention. In this regard, each block in the flowchart or block diagram may represent one module, one program segment, or a part of code, where the module, the program segment, or the part of code includes one or more executable instructions used for implementing specified logic functions. It should also be noted that, in some alternative implementations, functions marked in the blocks may also occur in a sequence different from the sequence marked in the drawing. For example, two consecutive blocks actually can be executed in parallel substantially, and sometimes, they can also be executed in reverse order, which depends on the functions involved. Each block in the block diagram and/or flowchart, and a combination of blocks in the block diagram and/or flowchart, may be implemented by a dedicated hardware-based system for executing corresponding functions or operations, or may be implemented by a combination of dedicated hardware and computer instructions.

As will be understood by those skilled in the art, embodiments of the present disclosure may be embodied as a method, a system or a computer program product. Accordingly, embodiments of the present disclosure may take the form of an entirely hardware embodiment, an entirely software embodiment or an embodiment combining software and hardware for allowing specialized components to perform the functions described above. Furthermore, embodiments of the present disclosure may take the form of a computer program product embodied in one or more tangible and/or non-transitory computer-readable storage media containing computer-readable program codes. Common forms of non-transitory computer readable storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM or any other flash memory, NVRAM, a cache, a register, any other memory chip or cartridge, and networked versions of the same.

Embodiments of the present disclosure are described with reference to flow diagrams and/or block diagrams of methods, devices (systems), and computer program products according to embodiments of the present disclosure. It will be understood that each flow and/or block of the flow diagrams and/or block diagrams, and combinations of flows and/or blocks in the flow diagrams and/or block diagrams, can be implemented by computer program instructions. These computer program instructions may be provided to a processor of a computer, an embedded processor, or other programmable data processing devices to produce a special purpose machine, such that the instructions, which are executed via the processor of the computer or other programmable data processing devices, create a means for implementing the functions specified in one or more flows in the flow diagrams and/or one or more blocks in the block diagrams.

These computer program instructions may also be stored in a computer-readable memory that can direct a computer or other programmable data processing devices to function in a particular manner, such that the instructions stored in the computer-readable memory produce a manufactured product including an instruction means that implements the functions specified in one or more flows in the flow diagrams and/or one or more blocks in the block diagrams.

These computer program instructions may also be loaded onto a computer or other programmable data processing devices to cause a series of operational steps to be performed on the computer or other programmable devices to produce processing implemented by the computer, such that the instructions (which are executed on the computer or other programmable devices) provide steps for implementing the functions specified in one or more flows in the flow diagrams and/or one or more blocks in the block diagrams. In a typical configuration, a computer device includes one or more Central Processors (CPUs), an input/output interface, a network interface, and a memory. The memory may include forms of a volatile memory, a random access memory (RAM), and/or non-volatile memory and the like, such as a read-only memory (ROM) or a flash RAM in a computer-readable storage medium. The memory is an example of the computer-readable storage medium.

The computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The computer-readable medium includes non-volatile and volatile media, and removable and non-removable media, wherein information storage can be implemented with any method or technology. Information may be modules of computer-readable instructions, data structures and programs, or other data. Examples of a non-transitory computer-readable medium include but are not limited to a phase-change random access memory (PRAM), a static random access memory (SRAM), a dynamic random access memory (DRAM), other types of random access memories (RAMs), a read-only memory (ROM), an electrically erasable programmable read-only memory (EEPROM), a flash memory or other memory technologies, a compact disc read-only memory (CD-ROM), a digital versatile disc (DVD) or other optical storage, a cassette tape, tape or disk storage or other magnetic storage devices, a cache, a register, or any other non-transmission media that may be used to store information capable of being accessed by a computer device. The computer-readable storage medium is non-transitory, and does not include transitory media, such as modulated data signals and carrier waves.

The specification has described attention monitoring methods, apparatus, and systems for vehicle-based health monitoring. The illustrated steps are set out to explain the exemplary embodiments shown, and it should be anticipated that ongoing technological development will change the manner in which particular functions are performed. Thus, these examples are presented herein for purposes of illustration, and not limitation. For example, steps or processes disclosed herein are not limited to being performed in the order described, but may be performed in any order, and some steps may be omitted, consistent with the disclosed embodiments. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments.

While examples and features of disclosed principles are described herein, modifications, adaptations, and other implementations are possible without departing from the spirit and scope of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise.

It will be appreciated that the present invention is not limited to the exact construction that has been described above and illustrated in the accompanying drawings, and that various modifications and changes can be made without departing from the scope thereof. It is intended that the scope of the invention should only be limited by the appended claims.

What is claimed is:

1. A vehicle, comprising:
one or more sensors configured to capture occupant information of an occupant of the vehicle and environmental information; and
a processor coupled to the one or more sensors and configured to:
associate the captured occupant information and the captured environmental information with an identity of the occupant; and
transmit the captured occupant information and the captured environmental information together with the associated identity of the occupant to a third party device.

2. The system of claim 1, wherein the environmental information comprises past locations of the vehicle.

3. The system of claim 2, wherein the past locations are associated with food.

4. The system of claim 1, wherein the environmental information comprises a condition of the vehicle.

5. The system of claim 4, wherein the condition comprises information of an accident that the vehicle is involved.

6. The system of claim 1, wherein the environmental information comprises visual data of an environment outside the vehicle.

7. The system of claim 6, wherein:
to capture the occupant information, the processor is configured to capture a health parameter of the occupant; and
the processor is further configured to adjust the captured health parameter based on a baseline reading of the health parameter of an average person in response to the captured visual data.

8. The system of claim 1, wherein to capture the occupant information, the one or more sensors are configured to measure one or more health parameters of the occupant.

9. The system of claim 8, further comprising a user interface configured to receive an instruction identifying at least a part of the occupant's body, of which the one or more sensors are configured to measure the one or more health parameters.

10. The system of claim 1, wherein before associating the captured occupant information and the captured environmental information with the identity of the occupant, the processor is further configured to determine the identity of the occupant based on the captured occupant information.

11. The system of claim 1, wherein the third party device is associated with a health practitioner.

12. A vehicle-based health monitoring method, comprising:
capturing, by a vehicle, occupant information of an occupant of the vehicle;
capturing, by the vehicle, environmental information;
associating, by the vehicle, the captured occupant information and the captured environmental information with an identity of the occupant; and
transmitting, by the vehicle, the captured occupant information and the captured environmental information together with the associated identity to a third party device.

13. The method of claim 12, wherein the environmental information comprises past locations of the vehicle.

14. The method of claim 13, wherein the past locations are associated with food.

15. The method of claim 12, wherein the environmental information comprises a condition of the vehicle.

16. The method of claim 15, wherein the condition comprises information of an accident that the vehicle is involved.

17. The method of claim 12, wherein the environmental information comprises visual data of an environment outside the vehicle.

18. The method of claim 17, wherein capturing the occupant information comprises capturing a health parameter of the occupant; and
the method further comprising adjusting the captured health parameter based on a baseline reading of the health parameter of an average person in response to the captured visual data.

19. The method of claim 12, wherein capturing the occupant information comprises measuring one or more health parameters of the occupant.

20. A vehicle, comprising:
one or more sensors configured to capture one or more health parameters of an occupant of the vehicle and environmental information; and
a processor coupled to the one or more sensors and configured to:
determine an identity of the occupant;
associate the captured one or more health parameters and the captured environmental information with the determined identity; and
transmit the captured one or more health parameters and the captured environmental information with the associated identity to a third party device.
